(19) United States
(12) Patent Application Publication
(10) Pub. No.: US 2022/0135919 A1
(43) Pub. Date: May 5, 2022

(54) INTEGRATED BIOREACTOR SYSTEMS

(71) Applicant: King Abdullah University of Science and Technology, Thuwal (SA)

(72) Inventors: Valerio MAZZONE, Thuwal (SA); Marcella BONIFAZI, Thuwal (SA); Ananya ASHOK, Thuwal (SA); Ahmed AL DAWSARI, Thuwal (SA); Andrea FRATALOCCHI, Thuwal (SA)

(21) Appl. No.: 17/434,500

(22) PCT Filed: Feb. 26, 2020
(86) PCT No.: PCT/IB2020/051652

§ 371 Date: Aug. 27, 2021

Related U.S. Application Data
(60) Provisional application No. 62/811,819, filed on Feb. 28, 2019.
(60) Provisional application No. 62/942,818, filed on Dec. 3, 2019.

(51) Int. Cl. C12M 1/00; C12N 1/12; C12M 1/34; C12R 1/89

(52) U.S. Cl. CPC C12M 21/02; C12M 41/06; C12M 43/08; C12M 23/22; C12R 2001/89; C12M 41/12; C12M 23/18; C12M 23/38; C12N 1/12

(57) ABSTRACT

Embodiments of the present disclosure describe bioreactor systems that integrate phototrophic organism cultivation with energy harvesting, methods of using said bioreactor systems, and the like. The bioreactor systems can comprise a bioreactor, wherein the bioreactor is configured to cultivate a phototrophic organism in a liquid growth medium, and at least one transparent photovoltaic panel positioned between the bioreactor and a light source, where the transparent photovoltaic panel transmits select wavelengths of light and absorbs select wavelengths of light.

### BACKGROUND

Algae farming is a rapidly growing interest of governments and industries across a wide range of sectors, including, for example, the food, pharmaceutical and nutraceutical industries, as well as animal farming, acquariology and bio-fuel production. Algae farming is also a promising candidate as a carbon negative technology, allowing factories to reduce their environmental impact on global warming. However, there is still room for improving current technology to achieve greater CO₂ reduction, among other things.

### SUMMARY

Bioreactor systems that integrate phototrophic organism cultivation with energy harvesting are described herein.

In a first aspect, the present invention is directed to bioreactor systems comprising a bioreactor, wherein the bioreactor is configured to cultivate at least one species of phototrophic organisms in a growth medium, and at least one transparent photovoltaic panel positioned between the bioreactor and a light source, where the transparent photovoltaic panel transmits select wavelengths of light and absorbs select wavelengths of light. In certain embodiments, the transparent photovoltaic panel is configured to transmit select wavelengths of visible light, wherein the wavelengths of visible light transmitted are selected to match the requirements or meet the needs of the species of phototrophic organisms that is/are being cultivated. In certain embodiments, the transparent photovoltaic panel is configured to absorb infrared radiation, wherein the absorbed infrared radiation is harvested to produce electricity and/or the absorption of the infrared radiation reduces radiation heat transfer to the bioreactor. In certain embodiments, the transparent photovoltaic panel is configured to absorb ultraviolet light, wherein the absorption of ultraviolet light reduces photodamage to the phototrophic organism.

In another aspect, the present invention is directed to methods of using bioreactor systems comprising one or more of the following steps: exposing any of the bioreactor systems disclosed herein to a light source such that light from the light source is incident upon the transparent photovoltaic panel, wherein the transparent photovoltaic panel is configured to:

(a) transmit select wavelengths of visible light to the at least one species of phototropic organisms, wherein at least a portion of the transmitted visible light is used by the at least one species of phototrophic organisms for photosynthesis;

(b) absorb infrared radiation, wherein at least a portion of the absorbed infrared radiation is converted to electricity and the absorption of the infrared radiation reduces radiation heat transfer to the bioreactor; and/or

(c) absorb ultraviolet light, wherein the absorption of ultraviolet light reduces the occurrence and/or extent of photodamage to the at least one species of phototrophic organisms;

delivering electricity produced by the bioreactor system to one or more components of the bioreactor system, and cultivating the growth of at least one species of phototrophic organisms.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a schematic diagram of a system for cultivating at least one species of phototropic organism and generating electricity, according to one or more embodiments of the present disclosure.

FIGS. 2A-2C are schematic diagrams of a flat-panel bioreactor system for cultivating at least one species of phototropic organism and generating electricity, where (A) is a perspective view, (B) is a top view, and (C) is a side view of said bioreactor system, according to one or more embodiments of the present disclosure.

FIG. 3 is a schematic diagram showing a perspective view of a raceway-pond bioreactor system for cultivating at least one species of phototropic organism and generating electricity, according to one or more embodiments of the present disclosure.

FIG. 4 is a schematic diagram showing a perspective view of a tubular bioreactor system for cultivating at least one species of phototropic organism and generating electricity, according to one or more embodiments of the present disclosure.

FIG. 5 is a flowchart of a method of cultivating at least one species of phototrophic organisms and generating electricity, according to one or more embodiments of the present disclosure.

FIG. 6 is a graphical view showing the growth rate of algae grown under artificial LED illumination and algae grown under visible light transmitted through a transparent photovoltaic panel, according to one or more embodiments of the present disclosure.

### DETAILED DESCRIPTION

Bioreactor systems that integrate phototrophic organism cultivation with energy harvesting are described herein. The bioreactor systems generally comprise a bioreactor and one or more transparent photovoltaic panels. The bioreactor can be configured to cultivate at least one species of phototrophic organisms in a growth medium. The transparent photovoltaic panels can have tunable absorption and/or transmittance spectra. For example, the transparent photovoltaic panel can be characterized by an absorption spectrum configured to intercept select wavelengths of light that are or may be harmful to the phototrophic organisms present in the bioreactor. The transparent photovoltaic panel can also be characterized by a transmittance spectrum configured to permit the passage of select wavelengths of light required by the phototrophic organism. In addition, solar radiation, such as the infrared portion of the spectrum, can be harvested and exploited to produce electricity, thereby reducing the thermal heating load in the reactor, while supplying the power required to run additional equipment that is used for cultivation, such as aeration and monitoring equipment, among others. The bioreactor systems thus can provide significant CO₂ reduction by generating electricity to self-power all or at least some of the other components of the systems.

In some embodiments, the transparency of the transparent photovoltaic panels in the visible spectrum can be about 70% or greater. In addition, the intensity of the visible spectrum that passes through the panel can be sufficient for phototrophic organisms, such as algae, to grow. The use of transparent photovoltaic panels in the bioreactor systems disclosed herein can partially or totally eliminate the need for cooling systems because the harvested energy can come from the infrared region of the spectrum. The energy produced by the solar panel can then be used to self-power the bioreactor systems, or at least a portion thereof. While the ultraviolet region of the spectrum is generally responsible for photodamage to, for example, microalgae and inhibit their growth, the transparent photovoltaic panels can absorb all or part of the ultraviolet spectrum to avoid photodamage and thus allow faster growth of the phototrophic organisms. These and other advantages are disclosed below.

### Definitions

The terms recited below have been defined as described below. All other terms and phrases in this disclosure shall be construed according to their ordinary meaning as understood by one of skill in the art.

As used herein, the term “open system” refers to a system partially or fully exposed to an environment. The term “closed system” refers to a system that is generally not open or exposed to an environment.

As used herein, the term “bioreactor” generally refers to any structure, system, device, or apparatus capable of supporting a biologically active environment and/or cultivating at least one species of phototropic organisms. Examples of bioreactors include, but are not limited to, photobioreactors, stir-tank reactors, airlift reactors, pneumatically mixed reactors, fluidized bed reactors, fixed-film reactors, hollow-fiber reactors, rotary cell culture reactors, packed-bed reactors, macro- and micro-bioreactors, raceways or raceway ponds, ponds, lakes, natural reservoirs, and the like, or combinations thereof. In addition, one or more bioreactors can be connected in series or in parallel.

As used herein, the term “photobioreactor” refers to any bioreactor that can use a light source to cultivate phototropic organisms. Examples of photobioreactors include, but are not limited to, tubular photobioreactors, plate photobioreactors, horizontal photobioreactors, foil photobioreactors, porous substrate bioreactors, and the like, or combinations thereof.

As used herein, the term “phototropic organism” includes any organism capable of converting light energy to chemical energy. For example, the phototropic organisms include organisms that utilize light as a source of energy to carry out any cellular processes, such as cellular metabolic processes. The phototropic organisms can be selected from unicellular organisms, multicellular organisms, or combinations thereof. The phototropic organisms can be selected from engineered organisms, organisms that exist in nature (e.g., naturally-occurring organisms), or combinations thereof. The phototropic organisms include photosynthetic organisms, which includes any organism capable of absorbing light for use in photosynthesis. Examples of phototropic organism include, but are not limited to, protists, such as algae and euglena, bacteria, higher plants, plankton, and protozoa, among others.

As used herein, “visible light” refers to electromagnetic radiation with any wavelength or frequency in the visible region of the electromagnetic spectrum. The boundary between and within the regions of the electromagnetic spectrum (e.g., radio waves, microwaves, infrared, visible, ultraviolet, X-rays, gamma rays, etc.) are not precisely defined by, for example, a universally agreed-upon standard. Accordingly, any recognized, accepted, or reasonable range of wavelengths or frequencies known in the art can be used to characterize or describe “visible light.” For example, “visible light” can be characterized by a wavelength ranging from about 380 nm to about 700 nm, among other ranges. Visible light includes, for example, violet light, blue light, cyan light, green light, yellow light, orange light, and red light.

As used herein, “infrared radiation” refers to electromagnetic radiation with wavelengths or frequencies in the infrared region of the electromagnetic spectrum. The boundary between and within the regions of the electromagnetic spectrum (e.g., radio waves, microwaves, infrared, visible, ultraviolet, X-rays, gamma rays, etc.) are generally not universally agreed-upon and can vary depending on, for example, the standard used. Accordingly, any recognized, accepted, or reasonable range of wavelengths or frequencies known in the art can be used to characterize or describe “infrared radiation.” For example, “infrared radiation” can be characterized by a wavelength ranging from about 700 nm to about 3000 nm, among other ranges. Infrared radiation can also be divided into smaller regions within the infrared region. The term “infrared radiation” thus can include, but is not limited to, regions known in the art as near-infrared, short-wavelength infrared, mid-wavelength infrared, long-wavelength infrared, far-infrared, or combinations thereof.

As used herein, “ultraviolet light” refers to electromagnetic radiation with wavelengths or frequencies in the ultraviolet region of the electromagnetic spectrum. The boundary between and within the regions of the electromagnetic spectrum (e.g., radio waves, microwaves, infrared, visible, ultraviolet, X-rays, gamma rays, etc.) are generally not universally agreed-upon and can vary depending on, for example, the standard used. Accordingly, any recognized, accepted, or reasonable range of wavelengths or frequencies known in the art can be used to characterize or describe “ultraviolet light.” For example, “ultraviolet light” can be characterized by a wavelength ranging from about 10 nm to about 400 nm, among other ranges. Ultraviolet light can also be divided into sub-regions or sub-types within the ultraviolet region. The term “ultraviolet light” thus can include, but is not limited to, regions known in the art as ultraviolet A, ultraviolet B, ultraviolet C, near ultraviolet, middle ultraviolet, far ultraviolet, vacuum ultraviolet, and extreme ultraviolet, or combinations thereof.

As used herein, the term “light source” generally refers to any process, system, device, apparatus, or structure that emits photons. The term “light source” can include, but is not limited to, natural light (e.g., light from the sun or solar radiation) or artificial light (e.g., light from any source other than the sun). As used herein, “solar radiation” generally refers to radiant energy emitted by the sun.

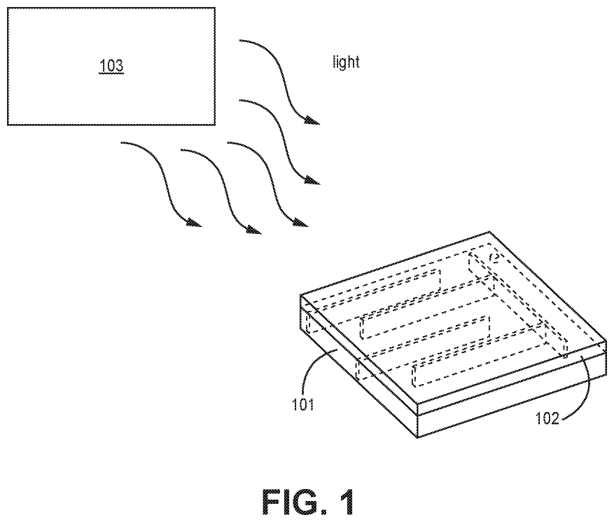

FIG. 1 is a schematic diagram of a bioreactor system, according to one or more embodiments of the present disclosure. As shown in FIG. 1, the bioreactor system 100 comprises a bioreactor 101 and one or more transparent photovoltaic panels 102 positioned between the bioreactor 101 and a light source 103. The bioreactor 101 optionally contains a growth medium and at least one species of phototrophic organisms. The one or more transparent photovoltaic panels 102 are configured to selectively absorb and/or transmit one or more wavelengths of light. In preferred embodiments, the transparent photovoltaic panels include one or more organic transparent photovoltaic panels (e.g., such as those available from YIRIS, Ubiquitus energy, Polysolar, and others that are commercially available).

The bioreactors can be open systems or closed systems. For example, in some embodiments, the bioreactors are open systems in which the bioreactors are exposed, or open, to an environment. In other embodiments, the bioreactors are closed systems in which the bioreactors are not exposed, or closed, to an environment. Examples of open and/or closed bioreactors include, but are not limited to, photobioreactors, stir-tank reactors, airlift reactors, pneumatically mixed reactors, fluidized bed reactors, fixed-film reactors, hollow-fiber reactors, rotary cell culture reactors, packed-bed reactors, macro- and micro-bioreactors, raceways, ponds, lakes, natural reservoirs, and the like, or combinations thereof. Examples of photobioreactors include, but are not limited to, tubular photobioreactors, plate photobioreactors, horizontal photobioreactors, foil photobioreactors, porous substrate photobioreactors, and the like, or combinations thereof. These shall not be limiting as other types of bioreactors can be used without departing from the scope of the present disclosure.

The one or more transparent photovoltaic panels are typically positioned between the light source and the bioreactor such that all or at least a portion (or a substantial portion) of the light from the light source is incident upon the one or more transparent photovoltaic panels. For example, in some embodiments, the one or more transparent photovoltaic panels are positioned between the light source and the bioreactor such that all or at least a substantial portion of the light that reaches the phototropic organisms present in the bioreactor is light that has passed through the one or more transparent photovoltaic panels. These embodiments may afford greater control over the cultivation conditions of the bioreactor. However, the positioning of the one or more transparent photovoltaic panels is not particularly limited so long as at least a portion of the light from the light source is incident upon at least a portion of the one or more of the transparent photovoltaic cells/panels.

The one or more transparent photovoltaic panels can also be positioned between the bioreactor and light source such that light from the light source irradiates the transparent photovoltaic panels at a select angle of incidence. As used herein, the term “angle of incidence” generally refers to the angle at which incident rays irradiate the surface of the transparent photovoltaic panel. The angle of incidence can affect the efficiency of the transparent photovoltaic panels and thus can be a useful consideration for the positioning of the transparent photovoltaic panels. In some embodiments, the angle of incidence is constant during operation of the bioreactor systems. In some embodiments, the angle of incidence is constant only for select durations. In some embodiments, the angle of incidence is transient during operation of the bioreactor systems or for select durations.

The angle of incidence is usually selected to maximize the efficiency (e.g., energy-harvesting efficiency) of the one or more transparent photovoltaic panels. Accordingly, in some embodiments, the angle of incidence is in the range of about 45 degrees to about 135 degrees, or any increment thereof. For example, in some embodiments, the angle of incidence is in the range of about 60 degrees to about 120 degrees. In some embodiments, the angle of incidence is in the range of about 75 degrees to about 105 degrees. In some embodiments, the angle of incidence is about 90 degrees. The angle of incidence, however, is not particularly limited and thus any angle of incidence can be used herein without departing from the scope of the present disclosure. For example, in some embodiments, the angle of incidence is in the range of about 0 degrees to about 180 degrees, or any increment thereof, for any duration.

In general, the absorption spectra and transmittance spectra, and optionally reflectance spectra, of the transparent photovoltaic panels are tunable. For example, the transparent photovoltaic panels can be fabricated or modified to exhibit the desired absorption and/or transmittance spectra (e.g., panels inkjet printed with one or more dyes to tune optical properties). The wavelengths of light selectively absorbed and/or transmitted by the transparent photovoltaic panels are not particularly limited and can include any wavelength of light or any ranges of wavelengths of light (e.g., wavelengths corresponding to one or more regions of the electromagnetic spectrum). Accordingly, in some embodiments, the transparent photovoltaic panels are configured to selectively absorb one or more wavelengths of light and transmit one or more wavelengths of light (e.g., other than the wavelengths of light that are absorbed). The selectively absorbed light can be a single wavelength of light or a range(s) of wavelengths of light. Similarly, the selectively transmitted light can be a single wavelength of light or range of wavelengths of light. In some embodiments, the wavelengths of light selectively absorbed and/or transmitted correspond to one or more regions or bands of the electromagnetic spectrum.

In some embodiments, the wavelengths of light absorbed and transmitted are selected to afford bioreactor systems with integrated capabilities, such as energy harvesting and organism cultivation. For example, in some embodiments, the transparent photovoltaic panels are configured to do one or more of the following: (a) absorb all or at least a portion of infrared radiation from a light source to generate electricity and optionally reduce radiation heat transfer to the bioreactor; (b) absorb all or at least a portion of ultraviolet light from the light source to reduce photodamage to the at least one species of phototropic organisms present in the bioreactor; and (c) transmit all or at least a portion of the visible light from the light source to the at least one species of phototrophic organisms. In this way, the bioreactor systems of the present disclosure can tune the wavelengths of infrared radiation and ultraviolet light absorbed to maximize the generation of electricity and minimize photodamage, respectively, while also tuning the wavelength of visible light transmitted to optimize the cultivation conditions to the needs of particular organisms.

While other wavelengths of radiation can be absorbed, the infrared radiation may be selected for absorption for any of a variety of reasons. For example, infrared radiation is generally not used by phototropic organisms, so absorbing the infrared radiation does not negatively affect the phototropic organisms in the bioreactor. Second, infrared radiation is responsible for much of the radiative heating of objects exposed to solar radiation (e.g., sunlight). Accordingly, reducing radiation heat transfer can result in significant reductions of heat transfer and thus alleviate the need entirely or at least partially for cooling systems. Finally, the sun's spectral output is such that more energy can be harvested from infrared radiation than can be harvested from other regions of the electromagnetic spectrum, such as ultraviolet and visible light, which is or can be required by the phototropic organisms.

In some embodiments, the one or more transparent photovoltaic panels absorb in the range of about 0% to about 100% of the incident infrared radiation. For example, in some embodiments, the one or more transparent photovoltaic panels are configured to absorb about 1% of the incident infrared radiation, about 10% of the incident infrared radiation, about 20% of the incident infrared radiation, about 30% of the incident infrared radiation, about 40% of the incident infrared radiation, about 50% of the incident infrared radiation, about 60% of the incident infrared radiation, about 70% of the incident infrared radiation, about 80% of the incident infrared radiation, about 90% of the incident infrared radiation, about 95% of the incident infrared radiation, about 99% of the incident infrared radiation, about 100% of the incident infrared radiation, or any increment thereof.

In some embodiments, the one or more transparent photovoltaic panels absorb in the range of about 0% to about 100% of the incident ultraviolet light. In some embodiments, the one or more transparent photovoltaic panels are configured to absorb 1% of the incident ultraviolet light, about 10% of the incident ultraviolet light, about 20% of the incident ultraviolet light, about 30% of the incident ultraviolet light, about 40% of the incident ultraviolet light, about 50% of the incident ultraviolet light, about 60% of the incident ultraviolet light, about 70% of the incident ultraviolet light, about 80% of the incident ultraviolet light, about 90% of the incident ultraviolet light, about 95% of the incident ultraviolet light, about 99% of the incident ultraviolet light, about 100% of the incident ultraviolet light, or any increment thereof.

In some embodiments, the one or more transparent photovoltaic panels transmit in the range of about 0% to about 100% of the incident visible light. In some embodiments, the one or more transparent photovoltaic panels are configured to transmit about 1% of the incident visible light, about 10% of the incident visible light, about 20% of the incident visible light, about 30% of the incident visible light, about 40% of the incident visible light, about 50% of the incident visible light, about 60% of the incident visible light, about 70% of the incident visible light, about 80% of the incident visible light, about 90% of the incident visible light, about 95% of the incident visible light, about 99% of the incident visible light, about 100% of the incident visible light, or any increment thereof.

As described above, in some embodiments, the absorbed infrared radiation is converted or used to generate electricity. Accordingly, in some embodiments, the bioreactor systems are characterized by an energy conversion efficiency of the transparent photovoltaic panels. In some embodiments, the energy conversion efficiency of the transparent photovoltaic panels is in the range of about 1% to about 45%, or any increment thereof. In some embodiments, the energy conversion efficiency of the one or more transparent photovoltaic panels is about 1%, about 5%, about 6%, about 7%, about 8%, about 9%, about 10%, about 15%, about 20%, about 25%, about 30%, about 35%, about 40%, about 45%, or any increment thereof. In some embodiments, the energy conversion efficiency of the one or more transparent photovoltaic panels is about 10%.

In some embodiments, the generated electricity is delivered to one or more components of the bioreactor system. The one or more components of the bioreactor system 100 are not particularly limited and can include any components known in the art that are used in connection with the operation of bioreactors and other related systems. In some embodiments, the one or more components include equipment relating to aeration of the algae medium and monitoring. In some embodiments, the one or more components include, in addition or in the alternative, pumps, heating systems, cooling systems, sensors, gauges, and/or waste water treatment systems, among other components. In some embodiments, the electricity generated from the absorbed infrared radiation is sufficient to power all of the components of the bioreactor system without an external power supply source (e.g., a power supply source other than the one or more transparent photovoltaic cells/panels of the present disclosure). In these embodiments, the bioreactor system is a self-powered bioreactor system.

In some embodiments, the absorption of infrared radiation reduces radiation heat transfer to the bioreactor. The absorption of infrared radiation thus can be used to modulate the temperature of the bioreactor. For example, in some embodiments, the absorption of incident infrared radiation reduces a temperature of the bioreactor (e.g., relative to a conventional bioreactor in which no infrared radiation is absorbed) by at least about 1° C. or more. For example, in some embodiments, the temperature is reduced by about 1° C., about 5° C., about 10° C., about 15° C., about 20° C., about 25° C., about 30° C., about 35° C., about 40° C., about 45° C., about 50° C., about 55° C., about 60° C., about 70° C., about 75° C., about 80° C., about 85° C., about 90° C., about 95° C., about 100° C., or any increment thereof. In some embodiments, the reduction in radiation heat transfer is sufficient to operate the bioreactor systems without any cooling systems. In some embodiments, the reduction in radiation heat transfer is sufficient to operate the bioreactor systems with a smaller cooling system than that which would otherwise be required in conventional bioreactors, which do not absorb infrared radiation.

In some embodiments, the absorption of ultraviolet light reduces photodamage to the at least one species of phototropic organisms. The reduction in photodamage can be characterized by the percentage of the population of the at least one species of phototropic organisms with photodamage. In some embodiments, the percentage of the population of the at least one species of phototropic organisms with photodamage is in the range of about 0% to about 50%, or any increment thereof. For example, in some embodiments, the percentage of the population of the at least one species of phototropic organisms with photodamage is less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 40%, less than about 50%, or any increment thereof.

In some embodiments, the transmitted visible light has wavelengths suitable for the one or more phototropic organisms present in the bioreactor. For example, in some embodiments, the transmitted visible light has wavelengths selected to optimize the cultivation conditions of the bioreactor to the needs of particular phototropic organisms. For example, in some embodiments, the transmitted visible light has a wavelength in the range of about 380 nm to about 740 nm, or any increment thereof. In some embodiments, the transmitted visible light has a wavelength in the range of about 500 nm to about 650 nm, or any increment thereof. In some embodiments, the transmitted visible light has a wavelength in the range of about 580 nm to about 650 nm.

The lifetimes of the one or more transparent photovoltaic panels can vary and depend on the particular transparent photovoltaic panel being used. In some embodiments, the lifetimes of the one or more transparent photovoltaic panels can be up to about 20 years. For example, in some embodiments, the lifetime of the one or more transparent photovoltaic panels can range from about 1 year to about 10 years. In some embodiments, the lifetime of the one or more transparent photovoltaic panels is about 1 year, about 2 years, about 3 years, about 4 years, about 5 years, about 6 years, about 7 years, about 8 years, about 9 years, about 10 years, or any increment thereof. In some embodiments, the lifetime of the one or more transparent photovoltaic panels is about 10 years or less, or about 15 years or less, among other lifetimes.

In certain embodiments, the bioreactor systems comprise a bioreactor with light-transparent walls and an organic transparent photovoltaic panel provided on a top side of the bioreactor. The bioreactor comprises algae that is grown in a liquid medium according to the requirements of the strain of choice. The absorption spectrum of the solar cell is configured to intercept ranges of wavelengths of light that are harmful to the algae, while letting pass through only radiation that matches algae growth requirements and/or requirements to undergo photosynthesis. In addition, the infrared part of the solar spectrum is harvested by the photovoltaic panel and exploited to produce electricity, reducing the thermal heating load in the reactor and supplying the power to run additional equipment essential to algae cultivation (monitoring, aeration, etc.). Other configurations are possible as described in greater detail herein.

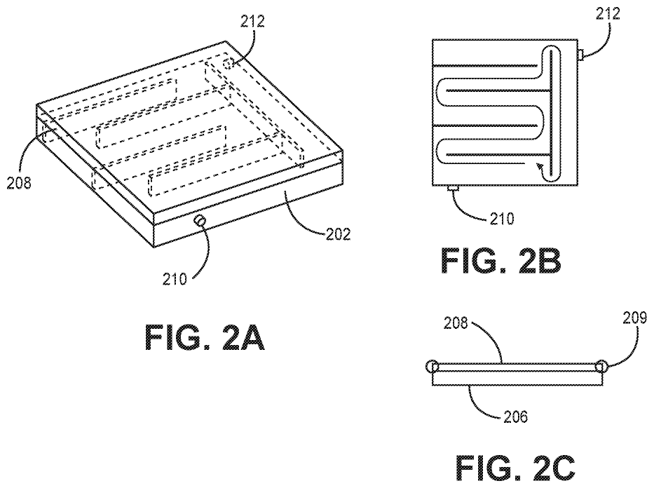

Non-limiting examples various configurations of bioreactor systems are provided in FIGS. 2-4 and discussed herein. For example, FIGS. 2A-2C is a schematic diagram of a flat-panel bioreactor system for cultivating at least one species of phototropic organism and generating electricity, according to one or more embodiments of the present disclosure. As shown in FIGS. 2A-2C, the flat bioreactor system can comprise a bioreactor 202 having an interior space partitioned into one or more reactor cells configured to receive and hold a growth medium 204, which can include at least one species of phototropic organisms 206; and a transparent photovoltaic panel 208 slidably coupled to the reactor container 202. In some embodiments, the bioreactor 202 comprises light-transparent walls with an organic transparent photovoltaic solar panel on a top side (e.g., such as YIRIS, which is commercially available). In some embodiments, the flat bioreactor system comprises a transparent photovoltaic panel that hermitically seals the bioreactor. In some embodiments, the flat bioreactor system is a self-powered bioreactor system, wherein the transparent photovoltaic panels harvest absorbed infrared radiation to generate electricity.

In some embodiments, the bioreactor 202 is a structure suitable for holding the growth media and the at least one species of phototropic organisms and for supporting a biologically active environment for the cultivation of the at least one species of phototropic organisms. In some embodiments, the bioreactor 202 is a transparent box. For example, in some embodiments, the bioreactor 202 comprises a bioreactor bottom coupled to two opposing sidewalls and two opposing endwalls. In some embodiments, the bioreactor 202 optionally further comprises one or more partitions for directing the fluid (e.g., the fluid including the growth medium and the at least one species of phototropic organisms) through the bioreactor. For example, in some embodiments, the partitions form one or more paths directing the fluid through the bioreactor in a continuous or non-continuous loop. In some embodiments, one or more of the sidewalls, endwalls, reactor bottom, and partitions are transparent (e.g., optically transparent) or selectively transmit wavelengths of light.

In some embodiments, the bioreactor system comprises a transparent photovoltaic panel as a bioreactor top. In some embodiments, the transparent photovoltaic panel is slidably coupled to the bioreactor 202. In some embodiments, the transparent photovoltaic panel is slidably coupled to the bioreactor 202 via casing 209. Advantageously, slidably coupled transparent photovoltaic panels promote ease of access to the transparent photovoltaic panel to facilitate the removal and replacement thereof. In some embodiments, the transparent photovoltaic panels hermetically seal the bioreactor 202 to afford a closed bioreactor system. In these embodiments, the hermetically sealed bioreactor 202 can provide greater control over the cultivation conditions, while also preventing alien objects and impurities from affecting the cultivation and growth of the at least one species of phototropic organisms, among other things.

In some embodiments, the bioreactors are provided in communication or fluid communication with one or more other components of the bioreactor systems. In some embodiments, the bioreactors are fluidly coupled to one or more inlet ports, such as inlet port 210. In some embodiments, the inlet ports include inlets for one or more of carbon dioxide, air, and other fluids used for promoting the cultivation and growth of the at least one species of phototropic organisms. In some embodiments, the bioreactors are provided in fluid communication with one or more outlet ports. For example, in some embodiments, the bioreactors are provided in fluid communication with an exhaust valve, such as exhaust valve 212. In some embodiments, the bioreactors 202 are provided in fluid communication with one or more of inlet ports, outlet ports, aeration systems, temperature control systems, filtration systems, harvesting systems, wastewater treatment systems, and fluid control systems, among other components and/or systems.

Accordingly, these shall not be limiting as other components and/or systems can be provided in fluid communication with the bioreactors without departing from the scope of the present disclosure.

FIG. 2B is a schematic diagram showing a top view of the flat-panel bioreactor system, according to one or more embodiments of the present disclosure. In some embodiments, the growth medium, phototrophic organisms, or both follow the path shown in FIG. 2B when air/CO₂ is pumped, fed or introduced into the bioreactor system 202. FIG. 2C is a schematic diagram of a side view of the flat-panel bioreactor system, according to one or more embodiments of the present disclosure. As shown, the box presents a casing which is designed to make the transparent photovoltaic panel slide along the top of the box and/or hermitically close the bioreactor 202. Although the transparent photovoltaic panels disclosed herein can be based on different technologies, their optical properties can be tuned during the fabrication stage in order to match the needs of, for example, the algae strain being cultivated.

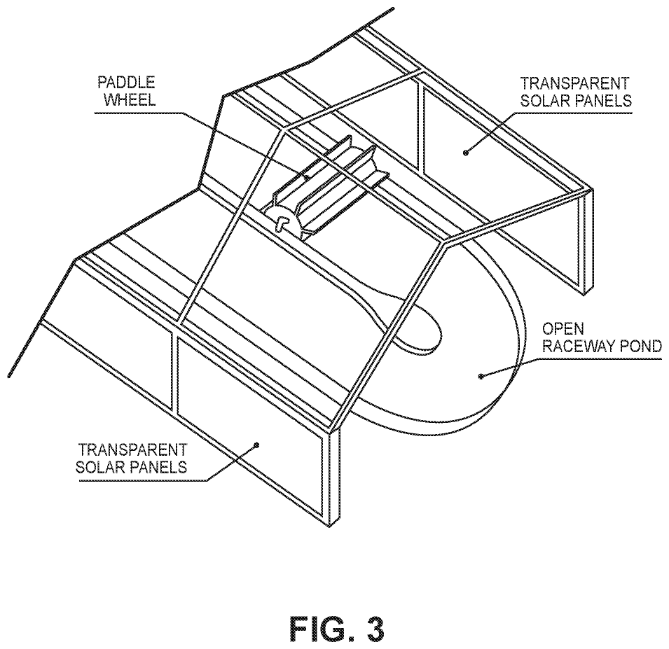

FIG. 3 is a schematic diagram of a raceway-pond bioreactor system for cultivating at least one species of phototropic organism and generating electricity, according to one or more embodiments of the present disclosure. As shown in FIG. 3, the race-way pond bioreactor system comprises a bioreactor comprising an open raceway pond and a structure comprising one or more transparent photovoltaic cells or panels, wherein the structure encloses the bioreactor. In some embodiments, the raceway pond bioreactor system further comprises a paddlewheel to promote fluid flow through the raceway pond.

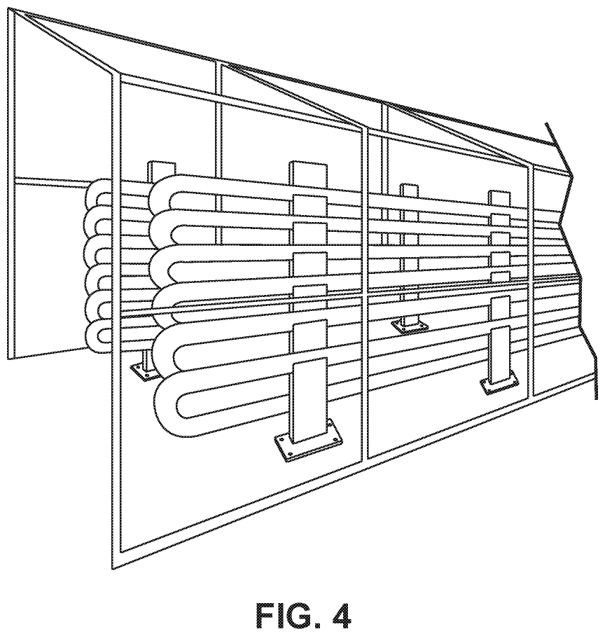

FIG. 4 is a schematic diagram of a tubular bioreactor system for cultivating at least one species of phototropic organism and generating electricity, according to one or more embodiments of the present disclosure. As shown in FIG. 4, the tubular bioreactor system comprises a tubular bioreactor and a structure comprising one or more transparent photovoltaic cells or panels, wherein the structure encloses the tubular bioreactor. In some embodiments, the tubular bioreactor is a reactor in which a growth medium containing at least one species of phototropic organisms flows through one or more pipes or tubes.

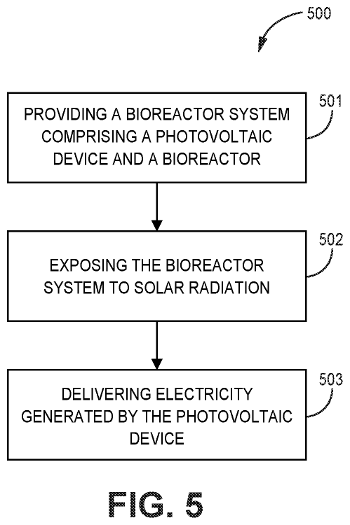

FIG. 5 is a flowchart of a method of cultivating at least one species of phototropic organisms and producing electricity, according to one or more embodiments of the present disclosure. As shown in FIG. 5, the method of cultivating phototrophic organisms can comprise one or more of the following steps: providing 501 a bioreactor system, exposing 502 the bioreactor system to a light source such as solar radiation, delivering 503 electricity from, or generated or produced by, the transparent photovoltaic panel or device to one or more components of the bioreactor system, and cultivating 504 the growth of at least one species of phototrophic organisms. In an embodiment, the method 500 can optionally further comprise tuning or adjusting an optical property (e.g., absorbance, transmittance, reflectance spectra, etc.) of the transparent photovoltaic panel.

The step 501 includes providing a bioreactor system. The bioreactor systems are not particularly limited. In some embodiments, the bioreactor systems include any of the bioreactor systems of the present disclosure. For example, in some embodiments, the bioreactor system comprises a bioreactor and a transparent photovoltaic panel positioned between a light source and the bioreactor. In some embodiments, the bioreactor comprises at least one species of phototrophic organisms and a liquid growth medium. In some embodiments, the transparent photovoltaic panel is an organic transparent photovoltaic panel or device or cell.

The one or more transparent photovoltaic panels can be placed in any position between the light source and the bioreactor. In some embodiments, the one or more transparent photovoltaic panels are positioned between the light source and the bioreactor such that at least a portion of the electromagnetic radiation emitted from the light source is incident upon at least a portion of the one or more transparent photovoltaic panels. In some embodiments, the one or more transparent photovoltaic panels are positioned between the light source and the bioreactor such that all the electromagnetic radiation emitted from the light source is incident upon at least a portion of the one or more transparent photovoltaic panels.

The step 502 includes exposing the bioreactor system to a light source such that light from the light source is incident upon the transparent photovoltaic panel. The bioreactor system can be exposed to electromagnetic radiation from any light source, including natural and/or non-natural, or artificial, light sources. In some embodiments, the exposing includes exposing to electromagnetic radiation of any wavelength and/or from any source. For example, in some embodiments, the exposing includes exposing to solar radiation from the sun. In some embodiments, the exposing includes exposing to artificial light, such as light from a light bulb or other such similar device. In some embodiments, the exposing includes irradiating. For example, in some embodiments, the exposing includes irradiating at least a portion of the bioreactor system. In some embodiments, the exposing includes irradiating at least a portion of the transparent photovoltaic panel to electromagnetic radiation.

Upon exposing the bioreactor system to the light source, the transparent photovoltaic panel can be configured to perform one or more of the following:

(a) transmit select wavelengths of visible light to the at least one species of phototropic organisms for their use in photosynthesis;

(b) absorb infrared radiation for use in producing electricity and/or to reduce radiation heat transfer to the bioreactor; and/or

(c) absorb ultraviolet light to reduce the occurrence and/or extent of photodamage to the at least one species of phototrophic organisms. In some embodiments, the transparent photovoltaic panel is configured to perform (a), (b), and (c). In some embodiments, the transparent photovoltaic panel is configured to perform two of (a), (b), and (c). In some embodiments, the transparent photovoltaic panel is configured to perform one of (a), (b), and (c). In other embodiments, the transparent photovoltaic panel is configured to at least absorb infrared radiation for use in the production of electricity.

The step 503 includes delivering the electricity produced in step (b)—that is, absorbing infrared radiation for use in the production of electricity—to one or more components of the bioreactor system. The delivering is not particularly limiting and can be performed according to any means or technique known in the art. In some embodiments, the electricity produced in step (b) is sufficient to power one or more components of the bioreactor system, such as aeration and/or monitoring equipment, among others. In some embodiments, the electricity produced in step (b) is sufficient to power the entire bioreactor system.

The step 504 includes cultivating the growth of the at least one species of phototrophic organisms. The cultivating is not particularly limited. For example, in some embodiments, the cultivating proceeds by supplying the resources required for the growth of the phototrophic organisms. In addition or in the alternative, in some embodiments, the cultivating proceeds by controlling the environment in which the phototrophic organisms is being cultivated. For example, in some embodiments, the cultivating can proceed by introducing/exhausting air/carbon dioxide to/from the bioreactor system. A person of ordinary skill in the art will appreciate and recognize other techniques that can be used to cultivate the at least one species of phototrophic organisms.

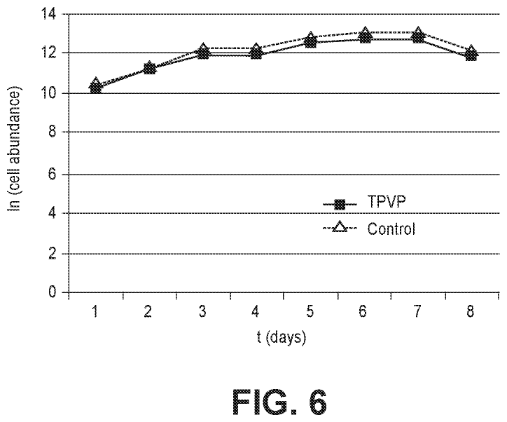

FIG. 6 is a graphical view showing the growth rate of algae, Dunaliella Salina, grown under artificial LED illumination and visible light transmitted through a transparent photovoltaic panel, according to one or more embodiments of the present disclosure. The transparent photovoltaic panel used was commercially available. Each point in the graph is the result of an average over three replicates of the same experiment. The initial culture medium for each replicate was about 100 mL. The experiment was run for about 8 days to follow the exponential growth phase of the algae.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

1. A bioreactor system, comprising:
a bioreactor, wherein the bioreactor is configured to cultivate at least one species of phototrophic organisms in a growth medium; and
one or more organic transparent photovoltaic panel positioned between the bioreactor and a light source;
wherein the organic transparent photovoltaic panel transmits select wavelengths of visible light and absorbs select wavelengths of non-visible light.

2. The bioreactor system according to claim 1, wherein the bioreactor has light transparent-walls.

3. The bioreactor system according to claim 1, wherein the organic transparent photovoltaic panel is configured to hermetically seal the bioreactor.

4. The bioreactor system according to claim 1, wherein the organic transparent photovoltaic panel is removably coupled to the bioreactor.

5. The bioreactor system according to claim 1, wherein the bioreactor is a raceway pond.

6. The bioreactor system according to claim 1, wherein the bioreactor is a tubular bioreactor.

7. The bioreactor system according to claim 1, wherein the select wavelengths of visible light comprise or consist of wavelengths of light that contribute to the growth of the phototrophic organism.

8. The bioreactor system according to claim 1, wherein the organic transparent photovoltaic panel transmits up to 70% of visible light.

9. The bioreactor system according to claim 1, wherein the organic transparent photovoltaic panel absorbs infrared radiation.

10. The bioreactor system according to claim 9, wherein the absorbed infrared radiation is used to produce electricity.

11. The bioreactor system according to claim 10, wherein the electricity that is produced is sufficient to power all other components of the bioreactor system.

12. The bioreactor system according to claim 1, wherein the absorption of infrared radiation reduces radiation heat transfer to the bioreactor.

13. The bioreactor system according to claim 1, wherein the bioreactor system is configured to control or maintain bioreactor temperatures without a cooling system.

14. The bioreactor system according to claim 1, wherein the organic transparent photovoltaic panel is configured to absorb ultraviolet light to reduce photodamage to the phototrophic organism.

15. The bioreactor system according to claim 1, wherein the transparent photovoltaic panel has a power conversion efficiency of at least about 10%.

16. The bioreactor system according to claim 1, wherein the transparent photovoltaic panel is positioned such that light irradiates the panel at an angle of about 90 degrees.

17. A method comprising cultivating phototrophic organisms using the bioreactor system according to claim 1.

18. A method of cultivating phototropic organisms, comprising:
providing a bioreactor system, wherein the bioreactor system comprises a transparent photovoltaic panel positioned between a light source and a bioreactor, wherein the bioreactor comprises at least one species of phototropic organisms and a liquid growth medium,
exposing the bioreactor system to a light source such that light from the light source is incident upon the transparent photovoltaic panel, wherein the transparent photovoltaic panel is configured to:
(a) transmit select wavelengths of visible light to the at least one species of phototropic organisms for their use in photosynthesis;
(b) absorb infrared radiation for use in producing electricity and to reduce radiation heat transfer to the bioreactor; and/or
(c) absorb ultraviolet light to reduce the occurrence and/or extent of photodamage to the at least one species of phototrophic organisms;
delivering the electricity produced in (b) to one or more components of the bioreactor system; and
cultivating the growth of the at least one species of phototrophic organisms.

19. The method according to claim 18, wherein the select wavelengths of visible light comprise or consist of wavelengths of light that contribute to the growth of the phototrophic organism.

20. The method according to claim 18, wherein the electricity produced in (b) is sufficient to power the entire bioreactor system.
